(19) United States
(12) Patent Application Publication
(10) Pub. No.: US 2023/0135671 A1
(43) Pub. Date: May 4, 2023

(54) USE OF BIOMARKERS IN THE TREATMENT OF FIBROTIC CONDITIONS

(71) Applicant: Boehringer Ingelheim International GmbH, Ingelheim am Rhein (DE)

(72) Inventors: Benjamin STROBEL, Ulm (DE); Patrick BAUM, Warthausen (DE); Claudia DIEFENBACH, Stockach (DE)

(21) Appl. No.: 17/914,373

(22) PCT Filed: Mar. 30, 2021
(86) PCT No.: PCT/EP2021/058285

§ 371 Date: Sep. 26, 2022

(30) Foreign Application Priority Data
Apr. 1, 2020 (EP) 20167596.4

(51) Int. Cl. C12Q 1/6883; A61K 31/4418; A61K 31/496; A61P 11/00; A61P 43/00

(52) U.S. Cl. CPC C12Q 1/6883; A61K 31/496; A61K 31/4418; A61P 11/00; A61P 43/00; C12Q 2600/158

(57) ABSTRACT

Genes selected from CEACAM6, CEACAM8, CTSG, DEFA4, LTF, MMP8, OLFM4, OLR1, SHISA4, ABCA13, EMID1, LMOD1, MPO, and PRTN3 can be used as biomarkers in methods for the treatment of progressive fibrosing interstitial lung diseases.

### FIELD OF THE INVENTION

This invention relates to antifibrotic agents, selected from nintedanib, its pharmaceutically acceptable salts, and pirfenidone, either as monotherapy or in combination with one another or in combination with sildenafil or a pharmaceutically acceptable salt thereof, for use in methods for the treatment of fibrotic disorders, selected from progressive fibrosing interstitial lung diseases, in a patient in need thereof comprising the monitoring of patient response to the treatment, the detection of the presence or absence of a beneficial response or the determination of the initiation of the treatment, each including the step of measuring in a biological sample from the patient the levels of expression of one or more biomarkers, selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof. In addition, the invention relates to the use of one or more of said biomarkers for selecting patients with said fibrotic disorders for treatment with antifibrotic agents, for predicting the progression of said fibrotic disorders in patients, or for determining whether an antifibrotic agent is efficacious in the treatment of said fibrotic disorders in a patient in need of treatment.

### BACKGROUND OF THE INVENTION

Progressive fibrosing ILD (PF-ILD) describes a disease behaviour observed in some patients across different Interstitial Lung Diseases (ILD). ILDs are a group of different diseases that affect the lace-like network of tissue and space around the air sacs of the lungs (interstitium). In some patients with ILD, there is a build-up of scar tissue in the lungs that keeps worsening. The lungs becoming progressively scarred, thickened and stiff leads to worsening of lung function, respiratory symptoms and worsening of health-related quality of life. Idiopathic pulmonary fibrosis (IPF) is considered the most typical example of PF-ILD.

Idiopathic pulmonary fibrosis (IPF) is a rare disease of unknown aetiology that is characterized by progressive fibrosis of the interstitium of the lung, leading to decreasing lung volume and progressive pulmonary insufficiency. The course of the disease in individual patients is variable: some patients progress rapidly, others have periods of relative stability punctuated by acute exacerbations and others progress relatively slowly. Acute exacerbations of IPF are events of respiratory deterioration of unidentified cause that occur in 5-10% of patients annually and are associated with a very poor outcome. IPF is most prevalent in middle aged and elderly patients, and usually presents between the ages of 40 and 70 years. The median life expectancy in IPF patients after diagnosis is 2 to 3 years. The latest update on clinical practice guideline for the treatment of IPF, jointly issued in 2015 by the American Thoracic Society (ATS), European Respiratory Society (ERS), Japanese Respiratory Society (JRS) and Latin American Thoracic Association (ALAT) has provided a conditional recommendation for treatment with nintedanib or pirfenidone for the majority of IPF patients, taking into account individual patient values and preferences. Conventional IPF treatments such as n-acetylcysteine (NAC), corticosteroids, cyclophosphamide, cyclosporine and azathioprine are not approved treatments for IPF, and their efficacy is questionable or they are even harmful. Nonpharmacological therapies such as pulmonary rehabilitation and long-term oxygen therapy are recommended for some patients, but their efficacy in patients with IPF has not been established. Lung transplant has been shown to positively impact survival in patients with IPF. Although the number of patients transplanted due to IPF has increased steadily over the last years, the scarce availability of donor organs, as well as the comorbidities and advanced age preclude many patients from referral to lung transplant.

In addition to IPF, PF-ILDs encompass i.a. those forms of the disease that are associated with scleroderma or systemic sclerosis (SSc-ILD) or with connective-tissue disease (CTD-ILD) or rheumatoid arthritis (RA-ILD). Nintedanib has been shown to provide efficacious treatments also for SSc-ILD and RA-ILD. PF-ILDs also comprise chronic fibrosing hypersensitivity pneumonitis (HP), idiopathic non-specific interstitial pneumonia (iNSIP), unclassifiable idiopathic interstitial pneumonia (IIP), environmental/occupational fibrosing lung disease, idiopathic pneumonia with autoimmune features (IPAF) and sarcoidosis.

Further fibrotic disorders for which treatments with nintedanib and/or pirfenidone have been considered include muscular dystrophies such as Duchenne muscular dystrophy, fibromatoses such as Dupuytren’s contracture and myelofibroses such as primary myelofibrosis (PMF).

Nintedanib (3-Z-[1-(4-(N-((4-methyl-piperazin-1-yl)-methylcarbonyl)-N-methyl-amino)-anilino)-1-phenyl-methylene]-6-methoxycarbonyl-2-indolinone), the compound of formula A,

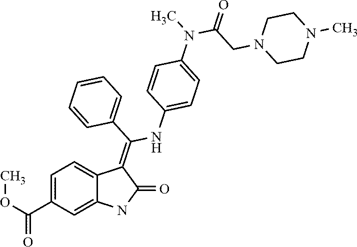 [SMILES: COC(=O)c1ccc2c(c1)NC(=O)/C2=C(\Nc1ccc(N(C)C(=O)CN2CCN(C)CC2)cc1)c1ccccc1]

is a highly potent, orally bioavailable, small molecule intracellular tyrosine kinase inhibitor. It inhibits vascular endothelial growth factor receptors (VEGFRs), platelet-derived growth factor receptors (PDGFRs) and fibroblast growth factor receptors (FGFRs). It binds competitively to the adenosine triphosphate (ATP) binding pocket of these receptors and blocks intracellular signalling. In addition, nintedanib inhibits Fms-like tyrosine-protein kinase 3 (Flt 3), lymphocyte-specific tyrosine-protein kinase (Lck), tyrosine-protein kinase lyn (Lyn) and proto-oncogene tyrosine-protein kinase src (Src) (Hilberg et al., Cancer Res. 2008, 68, 4774-4782). Thus, it has valuable pharmacological properties, e.g. for the treatment of oncological diseases, immunologic diseases or pathological conditions involving an immunologic component, or fibrotic diseases.

Nintedanib is described in WO 01/27081. WO 2004/013099 discloses its monoethanesulphonate salt which is especially suitable for development as a medicament; further salt forms are presented in WO 2007/141283. Pharmaceutical dosage forms comprising nintedanib are disclosed in WO 2009/147212 and in WO 2009/147220. The use of nintedanib for the treatment of immunologic diseases or pathological conditions involving an immunologic component is described in WO 2004/017948, the use for the treatment of oncological diseases is described in WO 2004/096224 and the use for the treatment of fibrotic diseases is described in WO 2006/067165.

Nintedanib has demonstrated anti-fibrotic and anti-inflammatory activity in preclinical models: Its antifibrotic potential of VEGFR, PDGFR, and FGFR inhibition with nintedanib has been evaluated in a series of preclinical studies. Data showing the kinase specificity profile of nintedanib has been published by Hilberg et al., Cancer Res. 2008; 68: 4774-82, i.e. regarding angiogenesis and fibrosis-related kinases such as VEGFR, PDGFR and FGFR, regarding Src family kinases related to inflammation and proliferation such as Src, Lck and Lyn, as well as other kinases, e.g. FLT-3, IGF1R, InsR, EGFR, HER2, CDK1, CDK2 and CDK4. Nintedanib was shown to inhibit PDGFR-a and-β activation and proliferation of normal human lung fibroblasts in vitro and to inhibit PDGF-BB-, FGF-2-, and VEGF-induced proliferation of human lung fibroblasts from patients with IPF and control donors. Nintedanib attenuated PDGF-or FGF-2-stimulated migration of lung fibroblasts from patients with IPF and inhibited transforming growth factor (TGF)-β-induced fibroblast to myofibroblast transformation of primary human lung fibroblasts from IPF patients (Hostettler et al., Respir. Res. 2014; 15: 157; Wollin et al., J. Pharmacol. Exp. Ther. 2014; 349:209-20). The polypharmacology of nintedanib has been described by L. Wollin et al., Eur. Respir. J. 2015; 45: 1434-1445. In two different mouse models of IPF, nintedanib exerted anti-inflammatory effects as shown by significant reductions in lymphocyte and neutrophil counts in the bronchoalveolar lavage fluid, reductions in inflammatory cytokines and reduced inflammation and granuloma formation in histological analysis of lung tissue. IPF mouse models also revealed nintedanib-associated antifibrotic effects as shown by significant reductions in total lung collagen and by reduced fibrosis identified in histological analyses.

The positive findings from preclinical investigations have also translated into the clinical setting: In multiple clinical trials, nintedanib has proven its ability to reduce the annual loss of lung function in IPF patients (i.a. INPULSIS, TOMORROW trials). Consequently, nintedanib has been approved for the treatment of IPF in numerous countries. Similarly, the disease progression could be slowed down in patients suffering from progressive lung fibroses other than IPF (INBUILD study) or from systemic scleroderma-associated interstitial lung disease (SSc-ILD) (SENSCIS study). Nintedanib is marketed for IPF under the brand name Ofev®. Its recommended dosage is 150 mg nintedanib twice daily. A lower dose of 100 mg twice daily dose is recommended to be used in patients who do not tolerate the 150 mg twice daily dose or in patients with mild hepatic impairment (Child Pugh A).

Pirfenidone (5-methyl-1-phenyl-2(1H)-pyridone), the compound of Formula B,

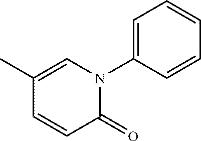

demonstrated anti-fibrotic activity in non-clinical models and efficacy in reducing the decline of the forced vital capacity (FVC) lung function in clinical trials (i.a. CAPACITY and ASCEND trials). Pirfenidone is marketed for the treatment of IPF as Esbriet® in capsules of 267 mg pirfenidone. The recommended daily dose for IPF patients is three capsules three times a day with food for a total of 2403 mg/day.

Upon initiating treatment, the dose should be titrated to the recommended daily dose of nine capsules per day over a 14-day period as follows:
Days 1 to 7: one capsule, three times a day (801 mg/day)
Days 8 to 14: two capsules, three times a day (1602 mg/day)
Day 15 onward: three capsules, three times a day (2403 mg/day)

Although nintedanib and pirfenidone can be considered a standard of care for patients diagnosed with IPF, it remains vital to predict, detect and monitor beneficial treatment responses to antifibrotic agents in order to provide the most efficient treatments possible. Thus, there is a need for improved means to follow the efficacy of treatment options against fibrotic diseases, identify patients that will most benefit from these treatments and to determine and adjust the therapies. The identification of suitable biomarkers to predict the clinical course of fibrotic disorders and benefits of therapy and thus to optimize the treatment for a given patient early in the course of the disease may help to fill this gap and remains one of the most relevant challenges in patient management.

Transcriptome-wide gene expression analysis represents a powerful approach for biomarker discovery as it has the potential to identify subtle treatment- or disease-dependent changes in relatively easy-to-obtain whole blood patient samples. To characterize transcriptional changes associated with the presence and extent of IPF, many studies have conducted microarray- or RNA-sequencing-based analyses using blood, peripheral blood mononuclear cells or lung tissue samples of human or murine origin (reviewed in Vukmirovic and Kaminski, Front Med (Lausanne), 2018 Apr 4;5:87). These studies usually identified several hundreds to thousands of genes that are altered in diseased (IPF) compared to healthy state. For instance, Yang et al. showed that 1428 genes were differentially expressed in the blood of mild IPF patients (Diffusing capacity for carbon monoxide (D_{L}CO) > 65%) compared to controls and 2790 transcripts were differentially expressed in severe IPF (D_{L}CO > 35%) compared to controls (Yang et al., PLoS One, 2012;7(6):e37708). Similarly, when comparing the lung tissue transcriptome of IPF patients with that of controls, Bauer et al. identified a total of 1,696 differentially expressed genes (Bauer et al., Am J Respir Cell Mol Biol. 2015 Feb;52(2):217-31.). The only gene set so far that has been validated across different patient cohorts is a set of 52 genes, identified by testing differentially expressed genes for their potential to predict transplantation-free survival (Herazo-Maya et al., Sci Transl Med. 2013 Oct 2;5(205):205ra136.). This gene signature was found enriched with genes associated with T-cell function. Its value for prediction of outcome (survival) in IPF patients has been validated across six different patient cohorts (Herazo-Maya et al., Lancet Respir Med. 2017 Nov;5(11):857-868.). Yet, in general, the overlap between genes identified in different studies is poor and it is unclear whether changes in the expression of any of those genes may indicate effective therapeutic treatment.

### SUMMARY OF THE INVENTION

In a first aspect, the present invention relates to an antifibrotic agent, in particular selected from nintedanib, its pharmaceutically acceptable salts, and pirfenidone, either as monotherapy or in combination with one another or in combination with sildenafil or a pharmaceutically acceptable salt thereof, for use in a method for the treatment of a fibrotic disorder, in particular selected from PF-ILDs, in a patient in need thereof, the method comprising the monitoring of patient response to the treatment including the steps
a) obtaining or having a first biological sample obtained from the patient prior to the start of administering the antifibrotic agent or providing or having said sample provided;
b) administering or having the antifibrotic agent administered to the patient;
c) obtaining or having a second biological sample obtained from the patient after administering the antifibrotic agent or providing or having said sample provided;
d) measuring in said first and second sample the levels of expression of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, or having said levels measured; and
e) comparing the levels of expression of said one or more biomarkers obtained from first and second biological samples or having said levels compared, optionally taking into account control values.

In a second aspect, the present invention relates to an antifibrotic agent, in particular selected from nintedanib, its pharmaceutically acceptable salts, and pirfenidone, either as monotherapy or in combination with one another or in combination with sildenafil or a pharmaceutically acceptable salt thereof, for use in a method for the treatment of a fibrotic disorder, in particular selected from PF-ILDs, in a patient in need thereof, the method comprising the detection of the presence or absence of a beneficial response including the steps
b) administering or having the antifibrotic agent administered to the patient;
c) obtaining or having a biological sample obtained from the patient after administering the antifibrotic agent or providing or having said sample provided;
d) measuring in said sample the levels of expression of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, or having said levels measured;
e) comparing or having the levels of expression of said one or more biomarkers compared with control values; and
f) determining or having determined whether or not the difference in the levels between the sample and the control reflects a beneficial response in the patient.

In a third aspect, the present invention relates to an antifibrotic agent, in particular selected from nintedanib, its pharmaceutically acceptable salts, and pirfenidone, either as monotherapy or in combination with one another or in combination with sildenafil or a pharmaceutically acceptable salt thereof, for use in a method for the treatment of a fibrotic disorder, in particular selected from PF-ILDs, in a patient in need thereof, the method comprising the determination of the initiation of the treatment including the steps
a) obtaining or having a biological sample obtained from the patient prior to the start of administering the antifibrotic agent or providing or having said sample provided;
d) measuring in said sample the levels of expression of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, or having said levels measured;
e) comparing or having the levels of expression of said one or more biomarkers compared with control values;
h) determining or having determined whether or not the difference between the expression levels in the sample and the control value reflects the need for initiation of a treatment of the fibrotic disorder; and
i) administering or having the antifibrotic agent administered to the patient if a need for initiation of a treatment of the fibrotic disorder has been determined.

In a forth aspect, the present invention relates to the use of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, for selecting patients with fibrotic disorders, in particular selected from PF-ILDs, for treatment with antifibrotic agents comprising
a) obtaining or having a biological sample obtained from the patient prior to the start of administering the antifibrotic agent or providing or having said sample provided;
d) measuring in said sample the levels of expression of said one or more biomarkers or having said levels measured;
e) comparing or having the levels of expression of said one or more biomarkers compared with control values;
j) determining or having determined whether the patient is eligible for treatment with antifibrotic agents on the basis of the results of the comparison.

In a fifth aspect, the present invention relates to the use of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, for predicting the progression of a fibrotic disorder, in particular selected from PF-ILDs, in a patient comprising
a) and/or c) obtaining or having a biological sample obtained from the patient prior to the start of administering an antifibrotic agent and/or after administering an antifibrotic agent or providing or having said sample provided;
d) measuring in said sample the levels of expression of said one or more biomarkers, or having said levels measured;
e) comparing or having the levels of expression of said one or more biomarkers compared with control values;
k) predicting or having predicted the progression of the disease on the basis of the results of the comparison.

In a sixth aspect, the present invention relates to the use of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, for determining whether an antifibrotic agent is efficacious in the treatment of a fibrotic disorder, in particular selected from PF-ILDs, in a patient in need of treatment comprising
a) obtaining or having a first biological sample obtained from the patient prior to the start of administering the antifibrotic agent or providing or having said sample provided;
b) administering or having the antifibrotic agent administered to the patient;
c) obtaining or having a second biological sample obtained from the patient after administering the antifibrotic agent or providing or having said sample provided;
d) measuring in said first and second sample the levels of expression of said one or more biomarkers or having said levels measured; and
e) comparing the levels of expression of said one or more biomarkers obtained from first and second biological samples or having said levels compared, optionally taking into account control values.

Further aspects of the present invention will become apparent to the person skilled in the art directly from the foregoing and following description and the examples.

### BRIEF DESCRIPTION OF THE DRAWINGS

FIGS. 1A-1H: Boxplots depicting the grouped changes in gene expression level (CPM, counts per million) for 8 genes out of the 14 selected Nintedanib-responsive genes as compared to Placebo at visit 2 (V2, baseline) and 12 weeks post treatment (V5) in IPF patients. FIG. 1A shows results for ABCA13; FIG. 1B shows results for CEACAM6, FIG. 1C shows results for CEACAM8; FIG. 1D shows results for CTSG; FIG. 1E shows results for DEFA4; FIG. 1F shows results for EMID1; FIG. 1G shows results for LMOD1; FIG. 1H shows results for LTF.

FIGS. 2A-2F: Boxplots depicting the grouped changes in gene expression level (CPM, counts per million) for the further 6 genes out of the 14 selected Nintedanib-responsive genes as compared to Placebo at visit 2 (V2, baseline) and 12 weeks post treatment (V5) in IPF patients. FIG. 2A shows results for MMP8; FIG. 2B shows results for MPO, FIG. 2C shows results for OLFM4; FIG. 2D shows results for OLR1; FIG. 2E shows results for PRTN3; FIG. 2F shows results for SHISA4.

FIG. 3: Boxplots depicting the patient-specific fold changes in gene expression for the 14 selected Nintedanib-responsive genes as compared to Placebo at visit 2 (V2, baseline) and 12 weeks post treatment (V5) in IPF patients. (TRT = treatment)

FIGS. 4A-4B: Pathway enrichment analysis was applied to functionally classify the 14 Nintedanib-responsive genes. FIG. 4A shows Gene Ontology (GO) Biological Pathway associations indicated by grey marks; FIG. 4B shows Reactome pathway associations indicated by grey marks.

FIG. 5: Unsupervised gene set variation analysis (GSVA) for the 14 Nintedanib-responsive genes. The boxplots show the GSVA score before (V2) and after treatment (V5, 12 weeks) with Nintedanib (N150mg_bid) or Placebo in IPF patients.

FIGS. 6A-6H: Boxplots depicting the grouped changes in gene expression level (CPM, counts per million) for 8 genes out of the 14 selected Nintedanib-responsive genes as compared to Placebo at visit 2 (V20, baseline), 24 weeks (V70) and 52 weeks post treatment (V90) in SSc-ILD patients. FIG. 6A shows results for ABCA13; FIG. 6B shows results for CEACAM6, FIG. 6C shows results for CEACAM8; FIG. 6D shows results for CTSG; FIG. 6E shows results for DEFA4; FIG. 6F shows results for EMID1; FIG. 6G shows results for LMOD1; FIG. 6H shows results for LTF.

FIGS. 7A-7F: Boxplots depicting the grouped changes in gene expression level (CPM, counts per million) for the further 6 genes out of the 14 selected Nintedanib-responsive genes as compared to Placebo at visit 2 (V20, baseline), 24 weeks (V70) and 52 weeks post treatment (V90) in SSc-ILD patients. FIG. 7A shows results for MMP8; FIG. 7B shows results for MPO, FIG. 7C shows results for OLFM4; FIG. 7D shows results for OLR1; FIG. 7E shows results for PRTN3; FIG. 7F shows results for SHISA4.

FIG. 8: Boxplots depicting the patient-specific fold changes in gene expression for the 14 selected Nintedanib-responsive genes as compared to Placebo at visit 2 (V2, baseline) and 24 weeks post treatment (V7) in SSc-ILD patients. (TRT = treatment)

FIG. 9: Unsupervised gene set variation analysis (GSVA) for the 14 Nintedanib-responsive genes. The boxplots show the GSVA score before (V20) and after treatment (V70, 24 weeks; V90, 52 weeks) with Nintedanib (Nint) or Placebo (Pbo) in SSc-ILD patients.

### DETAILED DESCRIPTION OF THE INVENTION

### General Terms and Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The terms “compound(s) according to this invention”, “compound(s) of formula (I)”, “compound(s) of the invention” and the like denote the compounds of the formula (I) according to the present invention including their tautomers, stereoisomers and mixtures thereof and the salts thereof, in particular the pharmaceutically acceptable salts thereof, and the solvates and hydrates of such compounds, including the solvates and hydrates of such tautomers, stereoisomers and salts thereof.

Also, unless specifically indicated, throughout the specification and the appended claims, a given chemical formula or name shall encompass tautomers and all stereo, optical and geometrical isomers (e.g. enantiomers, diastereomers, E/Z isomers etc...) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making organic or inorganic acid or base salts thereof. Examples of pharmaceutically acceptable salts include, but are not limited to, mineral or organic acid salts of basic residues such as amines; alkali or organic salts of acidic residues such as carboxylic acids; and the like.

Salts of other acids than those mentioned above which for example are useful for purifying or isolating the compounds of the present invention (e.g. trifluoro acetate salts) also comprise a part of the invention.

The terms “treatment” and “treating” as used herein embrace both therapeutic, i.e. curative and/or palliative, and preventive, i.e. prophylactic, treatment.

Therapeutic treatment (“therapy”) refers to the treatment of patients having already developed one or more of said conditions in manifest, acute or chronic form. Therapeutic treatment may be symptomatic treatment in order to relieve the symptoms of the specific indication or causal treatment in order to reverse or partially reverse the conditions of the indication or to stop or slow down progression of the disease.

Preventive treatment (“prevention”, “prophylaxis”) refers to the treatment of patients at risk of developing one or more of said conditions, prior to the clinical onset of the disease in order to reduce said risk.

The terms “treatment” and “treating” include the administration of one or more active compounds, in particular therapeutically effective amounts thereof, in order to prevent or delay the onset of the symptoms or complications and to prevent or delay the development of the disease, condition or disorder and/or in order to eliminate or control the disease, condition or disorder as well as to alleviate the symptoms or complications associated with the disease, condition or disorder.

When this invention refers to patients requiring treatment, it relates primarily to treatment in mammals, in particular humans.

The term “therapeutically effective amount” means an amount of a compound of the present invention that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

The term “biomarker” designates single genes or proteins and combinations of genes (e.g. “gene set”, “gene signature”) and/or of proteins, whose change in expression is indicative of a biological, pathological or pharmacological response to a medical treatment.

It is understood that all steps of a method described herein may be performed by only one single individual or entity or by more than one individuals or entities. Thus, whenever a method comprises performing a particular method step, this may equally relate to having said method step performed.

The present invention addresses the above-mentioned needs in the treatment of fibrotic disorders and provides biomarkers useful to this end. Thus, it allows for an efficient treatment of patients with fibrotic disorders by administering antifibrotic agents.

For underlying experimental findings, reference is made to the section Examples and Experimental Data.

In a first aspect of the present invention, it is found that biomarkers may be used to monitor the response of patients with fibrotic disorders to antifibrotic treatment.

Thus, the present invention relates to an antifibrotic agent, in particular selected from nintedanib, its pharmaceutically acceptable salts, and pirfenidone, either as monotherapy or in combination with one another or in combination with sildenafil or a pharmaceutically acceptable salt thereof, for use in a method for the treatment of a fibrotic disorder, in particular selected from PF-ILDs, in a patient in need thereof, the method comprising the monitoring of patient response to the treatment including the steps
a) obtaining or having a first biological sample obtained from the patient prior to the start of administering the antifibrotic agent or providing or having said sample provided;
b) administering or having the antifibrotic agent administered to the patient;
c) obtaining or having a second biological sample obtained from the patient after administering the antifibrotic agent or providing or having said sample provided;
d) measuring in said first and second sample the levels of expression of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, or having said levels measured; and
e) comparing the levels of expression of said one or more biomarkers obtained from first and second biological samples or having said levels compared, optionally taking into account control values.

The described method is likewise suitable for the detection of the presence or absence of a beneficial response in a patient.

Of course, as is evident to the one of skill in the art, the measurement of the biomarker expression level in the first sample, as defined in step d), does not necessarily need to be carried out after steps b) and c), but may be performed at any time after step a) and before step e). Hence, in this respect, the above sequence of method steps is not to be construed as a strict chronological order.

Optionally, the steps c), d) and e) may be repeated at multiple later time points during the treatment to obtain or provide further samples, measure their biomarker expression levels and compare them with previously obtained levels such that continuous monitoring of the patient response can be carried out.

Insofar, the method may equally be applied for the monitoring of patient compliance with a drug treatment protocol.

Likewise, the invention relates to a method for the treatment of a fibrotic disorder, in particular selected from PF-ILDs, in a patient in need thereof, the method comprising the monitoring of patient response to the treatment including said steps a), b), c), d), and e), wherein in step b) one or more antifibrotic agents, in particular selected from nintedanib, its pharmaceutically acceptable salts, and pirfenidone, either as monotherapy or in combination with one another or in combination with sildenafil or a pharmaceutically acceptable salt thereof, are administered to the patient.

Likewise, the invention relates to the use of one or more antifibrotic agents, in particular selected from nintedanib, its pharmaceutically acceptable salts, and pirfenidone, either as monotherapy or in combination with one another or in combination with sildenafil or a pharmaceutically acceptable salt thereof, in the manufacture of a medicament for the treatment of a fibrotic disorder, in particular selected from PF-ILDs, in a patient in need thereof, the method comprising the monitoring of patient response to the treatment including said steps a), b), c), d), and e).

In a second aspect of the present invention, it is found that biomarkers may be used to detect the presence or absence of a beneficial response of antifibrotic treatment in patients with fibrotic disorders.

Thus, the present invention relates to an antifibrotic agent, in particular selected from nintedanib, its pharmaceutically acceptable salts, and pirfenidone, either as monotherapy or in combination with one another or in combination with sildenafil or a pharmaceutically acceptable salt thereof, for use in a method for the treatment of a fibrotic disorder, in particular selected from PF-ILDs, in a patient in need thereof, the method comprising the detection of the presence or absence of a beneficial response including the steps
b) administering or having the antifibrotic agent administered to the patient;
c) obtaining or having a biological sample obtained from the patient after administering the antifibrotic agent or providing or having said sample provided;
d) measuring in said sample the levels of expression of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, or having said levels measured;
e) comparing or having the levels of expression of said one or more biomarkers compared with control values; and
f) determining or having determined whether or not the difference in the levels between the sample and the control reflects a beneficial response in the patient.

Insofar as the method relates to the detection of the presence of a beneficial response, it may equally be applied for the monitoring of patient compliance with a drug treatment protocol.

Likewise, the invention relates to a method for the treatment of a fibrotic disorder, in particular selected from PF-ILDs, in a patient in need thereof, the method comprising the detection of the presence or absence of a beneficial response including said steps b), c), d), e), and f), wherein in step b) one or more antifibrotic agents, in particular selected from nintedanib, its pharmaceutically acceptable salts, and pirfenidone, either as monotherapy or in combination with one another or in combination with sildenafil or a pharmaceutically acceptable salt thereof, are administered to the patient. Likewise, the invention relates to the use of one or more antifibrotic agents, in particular selected from nintedanib, its pharmaceutically acceptable salts, and pirfenidone, either as monotherapy or in combination with one another or in combination with sildenafil or a pharmaceutically acceptable salt thereof, in the manufacture of a medicament for the treatment of a fibrotic disorder, in particular selected from PF-ILDs, in a patient in need thereof, the method comprising the detection of the presence or absence of a beneficial response including said steps b), c), d), e), and f).

In a third aspect of the present invention, it is found that biomarkers may be used to determine the initiation of the antifibrotic treatment in patients with fibrotic disorders.

Thus, the present invention relates to an antifibrotic agent, in particular selected from nintedanib, its pharmaceutically acceptable salts, and pirfenidone, either as monotherapy or in combination with one another or in combination with sildenafil or a pharmaceutically acceptable salt thereof, for use in a method for the treatment of a fibrotic disorder, in particular selected from PF-ILDs, in a patient in need thereof, the method comprising the determination of the initiation of the treatment including the steps
a) obtaining or having a biological sample obtained from the patient prior to the start of administering the antifibrotic agent or providing or having said sample provided;
d) measuring in said sample the levels of expression of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, or having said levels measured;
e) comparing or having the levels of expression of said one or more biomarkers compared with control values;
h) determining or having determined whether or not the difference between the expression levels in the sample and the control value reflects the need for initiation of a treatment of the fibrotic disorder; and
i) administering or having the antifibrotic agent administered to the patient if a need for initiation of a treatment of the fibrotic disorder has been determined.

Likewise, the invention relates to a method for the treatment of a fibrotic disorder, in particular selected from PF-ILDs, in a patient in need thereof, the method comprising the determination of the initiation of the treatment including said steps a), d), e), h), and i) wherein in step i) one or more antifibrotic agents, in particular selected from nintedanib, its pharmaceutically acceptable salts, and pirfenidone, either as monotherapy or in combination with one another or in combination with sildenafil or a pharmaceutically acceptable salt thereof, are administered to the patient.

Likewise, the invention relates to the use of one or more antifibrotic agents, in particular selected from nintedanib, its pharmaceutically acceptable salts, and pirfenidone, either as monotherapy or in combination with one another or in combination with sildenafil or a pharmaceutically acceptable salt thereof, in the manufacture of a medicament for the treatment of a fibrotic disorder, in particular selected from PF-ILDs, in a patient in need thereof, the method comprising the determination of the initiation of the treatment including said steps a), d), e), h), and i).

In a fourth aspect of the present invention, it is found that biomarkers may be used to select patients with fibrotic disorders for treatment with antifibrotic agents.

Thus, the present invention relates to the use of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, for selecting patients with fibrotic disorders, in particular selected from PF-ILDs, for treatment with antifibrotic agents comprising
a) obtaining or having a biological sample obtained from the patient prior to the start of administering the antifibrotic agent or providing or having said sample provided;
d) measuring in said sample the levels of expression of said one or more biomarkers or having said levels measured;
e) comparing or having the levels of expression of said one or more biomarkers compared with control values;
j) determining or having determined whether the patient is eligible for treatment with antifibrotic agents on the basis of the results of the comparison.

Said use of biomarkers for patient selection may be applied, for instance, in a method of enriching a patient population for patients who are expected or who are not expected to show a beneficial response after treatment with an antifibrotic agent.

In a fifth aspect of the present invention, it is found that biomarkers may be used to predict the progression of fibrotic disorders in patients.

Thus, the present invention relates to the use of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, for predicting the progression of a fibrotic disorder, in particular selected from PF-ILDs, in a patient comprising
a) and/or c) obtaining or having a biological sample obtained from the patient prior to the start of administering an antifibrotic agent and/or after administering an antifibrotic agent or providing or having said sample provided;
d) measuring in said sample the levels of expression of said one or more biomarkers or having said levels measured;
e) comparing or having the levels of expression of said one or more biomarkers compared with control values;
k) predicting or having predicted the progression of the disease on the basis of the results of the comparison.

In a sixth aspect of the present invention, it is found that biomarkers may be used to assess the efficacy of an antifibrotic treatment in patients with fibrotic disorders.

Thus, the present invention relates to the use of one or more biomarkers, in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof, for determining whether an antifibrotic agent is efficacious in the treatment of a fibrotic disorder, in particular selected from PF-ILDs, in a patient in need of treatment comprising
a) obtaining or having a first biological sample obtained from the patient prior to the start of administering the antifibrotic agent or providing or having said sample provided;
b) administering or having the antifibrotic agent administered to the patient;
c) obtaining or having a second biological sample obtained from the patient after administering the antifibrotic agent or providing or having said sample provided;
d) measuring in said first and second sample the levels of expression of said one or more biomarkers or having said levels measured; and
e) comparing the levels of expression of said one or more biomarkers obtained from first and second biological samples or having said levels compared, optionally taking into account control values.

Of course, as is evident to the one of skill in the art, the measurement of the biomarker expression level in the first sample, as defined in step d), does not necessarily need to be carried out after steps b) and c), but may be performed at any time after step a) and before step e). Hence, in this respect, the above sequence of method steps is not to be construed as a strict chronological order.

According to one embodiment of any of the preceding aspects of the present invention, the antifibrotic agent is selected from nintedanib, its pharmaceutically acceptable salts, and pirfenidone, either as monotherapy or in combination with one another or in combination with sildenafil or a pharmaceutically acceptable salt thereof, preferably from nintedanib and its pharmaceutically acceptable salts, optionally in combination with sildenafil or sildenafil citrate, most preferably it is nintedanib or nintedanib monoethanesulphonate, optionally in combination with sildenafil citrate.

Suitable preparations for administering the active pharmaceutical ingredients of the present invention will be apparent to those with ordinary skill in the art and include for example tablets, pills, capsules, suppositories, lozenges, troches, solutions, syrups, elixirs, sachets, injectables, inhalatives and powders etc.. Suitable tablets may be obtained, for example, by mixing one or more of the above-mentioned active pharmaceutical ingredients with known excipients, for example inert diluents, carriers, disintegrants, adjuvants, surfactants, binders and/or lubricants.

In particular, pharmaceutical dosage forms for oral administration comprising nintedanib are disclosed in WO 2009/147212 and in WO 2009/147220. Suitable preparations for the administration of pirfenidone are, for instance, provided by WO 2007/038315 and WO 2017/172602.

For instance, therapeutically effective doses of nintedanib, when applied orally to a patient in need thereof, may be in the range from 100 mg to 300 mg per day, preferably twice daily application of 50 mg, 100 mg or 150 mg, approximately 12 hours apart.

The actual therapeutically effective amount or therapeutic dosage will of course depend on factors known by those skilled in the art such as age and weight of the patient, route of administration and severity of disease. In any case, the active compound will be administered at dosages and in a manner which allows a therapeutically effective amount to be delivered based upon a patient’s unique condition. Likewise, the determination of the necessity of dose adjustments, e.g. due to adverse reactions to the active pharmaceutical ingredient, and their putting into practice will be known to the one skilled in the art.

Thus, according to one embodiment, the antifibrotic agent is administered in the form of a pharmaceutical composition that comprises the antifibrotic agent and one or more pharmaceutically acceptable excipients.

According to another embodiment, the pharmaceutical composition is selected from compositions for oral administration, preferably from capsules and tablets, most preferably from capsules.

According to one embodiment, the fibrotic disorder is selected from progressive fibrosing interstitial lung diseases (PF-ILD), in particular diseases with a lung-fibrotic manifestation, such as idiopathic pulmonary fibrosis (IPF), systemic sclerosis-associated ILD (SSc-ILD), connective tissue disease-associated ILD (CTD-ILD), rheumatoid arthritis-associated ILD (RA-ILD), chronic fibrosing hypersensitivity pneumonitis (HP), idiopathic non-specific interstitial pneumonia (iNSIP), unclassifiable idiopathic interstitial pneumonia (IIP), environmental/occupational fibrosing lung disease, idiopathic pneumonia with autoimmune features (IPAF) and sarcoidosis;

preferably from IPF, SSc-ILD and RA-ILD.

According to another embodiment, the fibrotic disorder is selected from the group consisting of muscular dystrophies, fibromatoses and myelofibroses, preferably from Duchenne muscular dystrophy, Dupuytren’s contracture and primary myelofibrosis (PMF).

According to one embodiment, at the start of administration of the antifibrotic agent, the patient shows a forced vital capacity (FVC) of not more than about 40% of predicted normal, of about 40% to about 50% of predicted normal, of about 50% to about 60% of predicted normal, of about 60% to about 80% of predicted normal, or of not less than about 80% of predicted normal.

According to another embodiment, at the start of administration of the antifibrotic agent, the patient shows a diffusing capacity of lung for carbon monoxide (D_{L}CO) of not more than about 40% of predicted normal, of about 40% to about 60% of predicted normal, of about 60% to about 80% of predicted normal, or of not less than about 80% of predicted normal.

According to one embodiment, the biological sample in step c) is or has been obtained not earlier than about 1 week after the start of step b), e.g. within a time frame of about 1 week to about 52 preferably within a time frame of about 1 week to about 24 weeks after the start of step b),
more preferably after about 1, 2, 3, 4, 6, 8, 10, 12, 16, 20 or 24 weeks after the start of step b),
most preferably after about 1, 2, 4, 8 or 12 weeks after the start of step b).

According to another embodiment, the biological sample is a blood, peripheral blood mononuclear cells (PBMCs) or skin sample, preferably a blood or PBMC sample.

According to another embodiment, the biological sample is or has been obtained by blood taking or biopsy, preferably by blood taking.

According to one embodiment, the one or more biomarkers are genes associated with neutrophils, extracellular matrix and immunological responses,
in particular selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof,
preferably selected from the group consisting of CEACAM6, CEACAM8, CTSG, DEFA4, LMOD1, LTF, MMP8, OLFM4, OLR1, SHISA4 and combinations thereof,
more preferably selected from the group consisting of CEACAM6, CEACAM8, CTSG, DEFA4, LTF, MMP8, OLFM4, OLR1 and combinations thereof or from the group consisting of CEACAM6, CTSG, DEFA4, LTF, MMP8, OLFM4, and combinations thereof,
most preferably selected from the group consisting of CEACAM6, CTSG, DEFA4, LTF, OLFM4 and combinations thereof.

According to another embodiment, in steps d) and e) of the methods and uses described above, one single biomarker is employed which is a single gene selected from any of the above-mentioned groups of genes.

According to another embodiment, in steps d) and e) of the methods and uses described above, two or more biomarkers are employed which are single genes selected from any of the above-mentioned groups of genes,
in particular 2, 3, 4, 5, 6, 7, 8, 9, 10, 11, 12, 13 or 14 genes thereof, in more particular 2, 3, 5, 6, 8, 10 or 14 genes thereof,
preferably the 5 genes CEACAM6, CTSG, DEFA4, LTF, OLFM4 are employed or
the 6 genes CEACAM6, CTSG, DEFA4, LTF, MMP8, OLFM4 or
the 8 genes CEACAM6, CEACAM8, CTSG, DEFA4, LTF, MMP8, OLFM4, and OLR1 are employed, or
the 10 genes CEACAM6, CEACAM8, CTSG, DEFA4, LMOD1, LTF, MMP8, OLFM4, OLR1, SHISA4 or
the 14 genes CEACAM6, CEACAM8, CTSG, DEFA4, LTF, MMP8, OLFM4, OLR1, SHISA4, ABCA13, EMID1, LMOD1, MPO, and PRTN3.

According to another embodiment, in steps d) and e) of the methods and uses described above, the one or more biomarkers are combinations of one or more genes selected from any of the above-mentioned groups of genes,
in particular combinations of 2, 3, 4, 5, 6, 7, 8, 9, 10, 11, 12, 13 or 14 genes thereof, in more particular of 2, 3, 5, 6, 8, 10 or 14 genes thereof,
preferably a combination of the 5 genes CEACAM6, CTSG, DEFA4, LTF, OLFM4 is employed or
a combination of the 6 genes CEACAM6, CTSG, DEFA4, LTF, MMP8, OLFM4 or
a combination of the 8 genes CEACAM6, CEACAM8, CTSG, DEFA4, LTF, MMP8, OLFM4, and OLR1 are employed, or a combination of the 10 genes CEACAM6, CEACAM8, CTSG, DEFA4, LMOD1, LTF, MMP8, OLFM4, OLR1, SHISA4 or
a combination of the 14 genes CEACAM6, CEACAM8, CTSG, DEFA4, LTF, MMP8, OLFM4, OLR1, SHISA4, ABCA13, EMID1, LMOD1, MPO, and PRTN3.

According to another embodiment, the levels of biomarkers are determined by RNA sequencing or quantitative real-time PCR (such as but not limited to TaqMan gene expression assays or Low Density Array (TLDA) cards) or Nanostring nCounter technology (Geiss et al., Nat Biotechnol. 2008 Mar;26(3):317-25) or another RNA- or cDNA-based assay, preferably by RNA-sequencing or by quantitative real-time PCR, more preferably by quantitative real-time PCR.

According to one embodiment, the control value, in particular according to the first, second and sixth aspect of the invention, is determined using samples taken from the patient prior to the start of administering the antifibrotic agent, taken from patients treated with a placebo or taken from patients treated with another antifibrotic drug, e.g. other than Nintedanib, preferably using a sample taken from the patient prior to the start of administering the antifibrotic agent.

According to another embodiment, the control value, in particular according to the third, fourth, fifth and sixth aspect of the invention, is determined using samples taken from subjects that do not suffer from a fibrotic disorder or taken from subjects that are known to suffer from a fibrotic disorder.

According to another embodiment, the control value, in particular according to any aspect of the invention, is determined using samples taken from subjects that suffer from a fibrotic disorder with a progressive course of disease or taken from subjects that suffer from a fibrotic disorder with a stable course of disease.

The classification of progressive and stable courses of disease may depend on the circumstances of each individual case, but will be part of a routine process for the experienced practitioner in the field. For instance, progression of PF-ILDs may be defined as an absolute decrease of more than 10% of FVC and/or of more than 15% of D_{L}CO during serial pulmonary function tests within 6 or 12 months.

According to one embodiment, a measured biomarker expression level is considered, within the comparison according to step e), to be different from a control value, i.e. higher or lower than the control value, if their difference is at least about 10%, 15%, 20%, 25%, 30%, 35%, 40%, 45%, 50%, 55%, 60%, 65%, 65%, 70%, 75%, 80%, 85%, 90%, or 95% in relation to the control value, preferably if their difference is at least about 20%, 30%, 40% or 50%.

According to another embodiment, the presence of a beneficial response in the patient is indicated by the measured biomarker expression level in the sample being lower than the control value, and accordingly the absence of a beneficial response in the patient is indicated by the measured biomarker expression level in the sample being not lower than the control value,

e.g. when the control value is determined using samples taken from the patient prior to the start of administering the antifibrotic agent, taken from patients treated with a placebo or taken from subjects that suffer from a fibrotic disorder with a progressive course of disease.

According to another embodiment, the initiation of antifibrotic treatment is indicated or a patient with a fibrotic disorder is selected for antifibrotic treatment on the basis of the measured biomarker expression level in the sample being higher than the control value,

e.g. when the control value is determined using samples taken from subjects that do not suffer from a fibrotic disorder or taken from subjects that suffer from a fibrotic disorder with a stable course of disease.

According to another embodiment, the initiation of antifibrotic treatment is indicated or a patient with a fibrotic disorder is selected for antifibrotic treatment on the basis of the measured biomarker expression level in the sample being not lower than the control value,

e.g. when the control value is determined using samples taken from subjects that are known to suffer from a fibrotic disorder or taken from subjects that suffer from a fibrotic disorder with a progressive course of disease.

According to another embodiment, the efficacy of antifibrotic treatment is indicated by the measured biomarker expression level in the sample being lower than the control value,

e.g. when the control value is determined using samples taken from the patient prior to the start of administering the antifibrotic agent, taken from patients treated with a placebo or taken from subjects that suffer from a fibrotic disorder with a progressive course of disease.

According to another embodiment, the efficacy of antifibrotic treatment is indicated by the measured biomarker expression level in the sample being not higher than the control value,

e.g. when the control value is determined using samples taken from subjects that do not suffer from a fibrotic disorder, taken from subjects that suffer from a fibrotic disorder with a stable course of disease or taken from patients treated with another antifibrotic drug.

Of course, the comparison of levels of expression of biomarkers with control values or with one another according to step e) as well as the subsequent method steps f) to k), as applicable, may comprise the use of appropriate statistical considerations. Such methods, e.g. the use of hypothesis tests, confidence intervals and the like, and their application are well known to the one skilled in the art.

It is further found that the use of the above-mentioned biomarkers provides options to determine the need for the modification of the treatment of fibrotic disorders with antifibrotic agents. For instance, the detection of a beneficial response in a patient, according to the second aspect of the invention, provides further treatment options: The administration of the antifibrotic agent may be continued without changes of the dosage regiment if no intolerable side effects have been observed. Alternatively, if, despite a beneficial response, the side effects of the antifibrotic treatment are perceived as intolerable, the dose or the dose frequency may be reduced with the goal to minimize side effects while maintaining the beneficial treatment response.

Thus, according to one embodiment, the method further comprises the step
g1) continuing or having the administration of the antifibrotic agent to the patient continued,
if a beneficial response in the patient has been detected.

According to another embodiment, the method further comprises the step
g2) reducing or having the dose or dose frequency reduced;
if a beneficial response in the patient has been detected.

On the other hand, if no beneficial response could be detected and no intolerable side effects have occurred, either the discontinuation of the administration of the antifibrotic agent may be considered or the dose or dose frequency of the antifibrotic agent may be increased with the goal to achieve a beneficial response while keeping side effects at a tolerable level.

Thus, according to one embodiment, the method further comprises the step
g3) discontinuing or having discontinued the administration of the antifibrotic agent to the patient,
if no beneficial response in the patient has been detected.

According to another embodiment, the method further comprises the step
g4) increasing or having increased the dose or dose frequency of the antifibrotic agent,
if a beneficial response in the patient has been detected.

The same considerations apply mutatis mutandis to the response monitoring according to the first aspect of the invention.

The extent of said treatment modifications depend, of course, on the facts and circumstances of each individual case, but will be part of the routine of an experienced practitioner in the field of the invention.

### EXAMPLES AND EXPERIMENTAL DATA

The following examples are for the purpose of illustration of the invention only and are not intended in any way to limit the scope of the present invention.

### A) Clinical Trial to Assess the Effect of Nintedanib in Patients with IPF

A 12-week, double blind, randomised, placebo controlled, parallel group trial (followed by a single active arm phase of 40 weeks) evaluating the effect of nintedanib on biomarkers in patients with IPF and limited forced vital capacity (FVC) impairment.

Main Inclusion criteria: Male or female patients (40 years or older) with IPF diagnosis; FVC ≥ 80% predicted of normal at Visit 1 (screening)

Posology: Twice daily oral application of 150 mg nintedanib.

Primary Endpoint: Rate of change (slope) in FVC from baseline to week 52

Key Secondary Endpoint: Proportion of patients with disease progression as defined by absolute FVC (% predicted) decline ≥ 10% or death until week 52.

Safety criteria: Adverse events (especially SAE and other significant AE), physical examination, weight measurements, 12 lead electrocardiogram, vital signs and laboratory evaluations.

Statistical methods: Random coefficient regression models for continuous endpoints, Log rank tests, Kaplan-Meier plots and Cox regressions for time to event endpoints, logistic regression models or other appropriate methods for binary endpoints.

On the basis of this example and along with publicly well available information on similar clinical trials, the one skilled in the art will find no difficulty to design and conduct related clinical trials directed towards other aspects of the present invention.

### B) Determination of Biomarkers

Whole blood samples were prospectively collected in clinical trials studying the effect of Nintedanib versus Placebo treatment in cohorts of IPF, SSc and PF-ILD patients, respectively.

### RNA Isolation and Sequencing

Blood was sampled into PAXgene RNA tubes (PreAnalytix) and RNA was extracted using the PAXgene Blood RNA kit. Quantity of total RNA was determined by direct absorbance measurement at 260 nm using a spectrophotometer. RNA sequencing libraries were prepared using the Illumina TruSeq Stranded Total RNA Library Prep Kit with Ribo-Zero Globin. Briefly, following removal of ribosomal RNA and globin-encoding mRNA, cleaved RNA fragments were reversely transcribed into first strand cDNA using reverse transcriptase and random primers followed by second strand cDNA synthesis using DNA Polymerase I and RNase H. Strand origin was kept by using Uracil instead of Thymin in the nucleotide mix for first strand cDNA synthesis. Following 3′-adenylation and index adapter ligation, cDNA products were purified and enriched by PCR to create the final cDNA library. Library quantity was determined using the Quant-it Pico Green dsDNA reagent and quality was checked by analyzing cDNA fragment size using an Agilent Bioanalyzer 2100 device. Following library dilution to 5 nM, samples were pooled for cluster generation. RNA sequencing was conducted on an Illumina HiSeq-3000 sequencer.

### Computational Analysis

Quality control: The raw data (single-end read sequences of the captured cDNA fragments) obtained for each sample were assessed for quality using FASTQC v0.11.2. For a successful gene expression analysis of a sample, approximately 50 million reads were required. Reads were mapped to the human reference genome hg38 (GRCh38 Ensembl v. 84) using STAR v2.5.2a. The mapped reads were assessed using RNA-SeQC v1.1.8, whereby mapping statistics (e.g. unique mapping rate, number of detected genes, proportion of reads mapping to protein coding genes) provided the final criteria to decide on usability of the data.

Gene expression intensities were represented as either read counts or TPM values and calculated based on Ensembl v84 gene annotations using subread featureCounts and RSEM version 1.2.31 respectively. Further steps included the use of bamUtil version 1.0.11 and samtools version 1.1 for intermediate calculations such as indexing of bam-files and duplicate read marking. Finally, PCA and hierarchical clustering analysis were used to identify outliers.

Differential Expression Analysis: Only genes for which at least one sample displayed a count per million (cpm) ≥ 1 were used in the subsequent analyses. Normalization factors to scale the samples based on the raw library sizes were calculated using the weighted trimmed mean of M-values (TMM) method using the default parameters of edgeR’s calcNormFactors function (Robinson and Oshlack, Genome Biol. 2010;11(3):R25.). After these pre-processing steps, the log2 counts per million along with associated weights for each observation were then calculated based on the normalized library sizes from the previous step using limma’s voom function (Law et al., Genome Biol. 2014 Feb 3;15(2):R29.). Correlations between paired measurements per patient were estimated by the duplicateCorrelation function. For each gene, a repeated measures linear regression model was utilized with treatment (Nintedanib or placebo), visit and treatment-visit interaction as fixed effect and patient as blocking factor. Hereby the weights for each observation were taken into account. All linear models were implemented using the Im Fit function of the limma package. Estimates for the mean log2 fold change between follow-up visit at week 12 (wk12) and baseline (bl) in each treatment arm was calculated as

$$\begin{matrix}\text{log2} {\text{FCNint}}_{\text{w12}} \text{vs} {\text{Nint}}_{\text{bl}} \text{=} \left(\text{log2} {\text{Nint}}_{\text{w12}} \text{-log2} {\text{Nint}}_{\text{bl}}\right) \\ \text{log2} {\text{FCPBO}}_{\text{w12}} \text{vs} {\text{PBO}}_{\text{bl}} \text{=} \left(\text{log2} {\text{PBO}}_{\text{w12}} \text{-} \text{log2} \text{} {\text{PBO}}_{\text{bl}}\right)\end{matrix}$$

Changes were quantified by log2-fold changes and associated False Discovery Rate (FDR) adjusted p-values according to Benjamini-Hochberg.

Gene set variance analysis (GSVA): The Bioconductor package Gene Set Variance Analysis version 1.3.2 was applied to score each sample individually for the activity of the 14 genes (LTF, CEACAM6, CTSG, OLFM4, MMP8, CEACAM8, DEFA4, OLR1, SHISA4, ABCA13, EMID1, LMOD1, MPO, and PRTN3). The GSVA function was executed with an expression matrix of the log2 Transcripts Per Million (TPM) with a pseudocount of 0.01 added to each value, the list of 14 genes, and the parameter mx.diff set to true. Statistical differences between the GSVA scores between the different treatment groups were tested using a simple linear model and moderated t-statistics computed by the limma package using an empirical Bayes shrinkage method (Law et al., Genome Biol. 2014 Feb 3;15(2):R29.). Identification of genes altered upon Nintedanib treatment in patients with IPF

The effect of Nintedanib on several biomarkers has been assessed in the INMARK study. In this study, whole blood samples were prospectively collected from IPF patients at baseline and 12 weeks following treatment with either Placebo or Nintedanib (orally administered, 150 mg b.i.d.): 230 and 116 samples were obtained from the placebo and active treatment cohort, respectively. Total RNA was isolated from these blood samples, quality controlled (QC) and analyzed by whole-transcriptome RNA sequencing to identify differentially expressed genes (DEGs) between baseline and follow-up visit at week 12 in each treatment arm as well as Placebo- and Nintedanib treatment at each visit. Following QC, 110 samples from the Nintedanib treatment arm and 217 samples from the placebo arm qualified for analysis. 19 protein-coding genes showed significant differences (adj. p-value <0.05) when gene expression was compared pre- and post-Nintedanib treatment. 18 of these genes were downregulated and one gene was upregulated. The top ten of downregulated genes (CEACAM6, CEACAM8, CTSG, DEFA4, LMOD1, LTF, MMP8, OLFM4, OLR1, SHISA4) were initially selected for further characterization. Of the remaining eight downregulated genes, four additional genes (ABCA13, EMID1, MPO, PRTN3) were selected based on their fold change and proposed biological role, as evident from literature and pathway association tools (see below).

For these selected 14 genes, Table 1 summarizes the linear fold changes in gene expression and Benjamini-Hochberg adjusted p-values under Nintedanib and Placebo treatment between visit 2 (V2, baseline) and visit 5 (V5, 12 weeks).

TABLE 1

| Gene | Ensembl ID (v84) | Nintedanib treatment (V5 vs. V2) | | Placebo treatment (V5 vs. V2) | |
|---|---|---|---|---|---|
| | | fold change | adjusted p-value | fold change | adjusted p-value |
| DEFA4 | ENSG00000164821 | -1.776 | 0.005 | -1.088 | 0.957 |
| LMOD1 | ENSG00000163431 | -1.745 | 0.009 | -1.037 | 0.971 |
| OLFM4 | ENSG00000102837 | -1.657 | 0.033 | -1.141 | 0.957 |
| CTSG | ENSG00000100448 | -1.635 | 0.010 | -1.079 | 0.957 |
| PRTN3 | ENSG00000196415 | -1.623 | 0.033 | -1.019 | 0.985 |
| LTF | ENSG00000012223 | -1.608 | 0.005 | -1.126 | 0.957 |
| MMP8 | ENSG00000118113 | -1.587 | 0.009 | -1.154 | 0.927 |
| CEACAM8 | ENSG00000124469 | -1.490 | 0.009 | -1.113 | 0.957 |
| SHISA4 | ENSG00000198892 | -1.478 | 0.009 | 1.044 | 0.966 |
| OLR1 | ENSG00000173391 | -1.475 | 0.014 | -1.101 | 0.957 |
| EMID1 | ENSG00000186998 | -1.451 | 0.009 | -1.053 | 0.957 |
| CEACAM6 | ENSG00000086548 | -1.451 | 0.037 | -1.097 | 0.957 |
| ABCA13 | ENSG00000179869 | -1.369 | 0.033 | -1.124 | 0.917 |
| MPO | ENSG00000005381 | -1.357 | 0.016 | -1.033 | 0.969 |

DEFA4: defensin alpha 4; LMOD1: leiomodin 1; OLFM4: olfactomedin 4; CTSG: cathepsin G; PRTN3: proteinase 3; LTF: lactotransferrin; MMP8: matrix metallopeptidase 8; CEACAM6/8: carcinoembryonic antigen related cell adhesion molecule 6/8; SHISA4: shisa family member 4; OLR1: oxidized low density lipoprotein receptor 1; EMID1: EMI domain containing 1; ABCA13: ATP binding cassette subfamily A member 13; MPO: myeloperoxidase

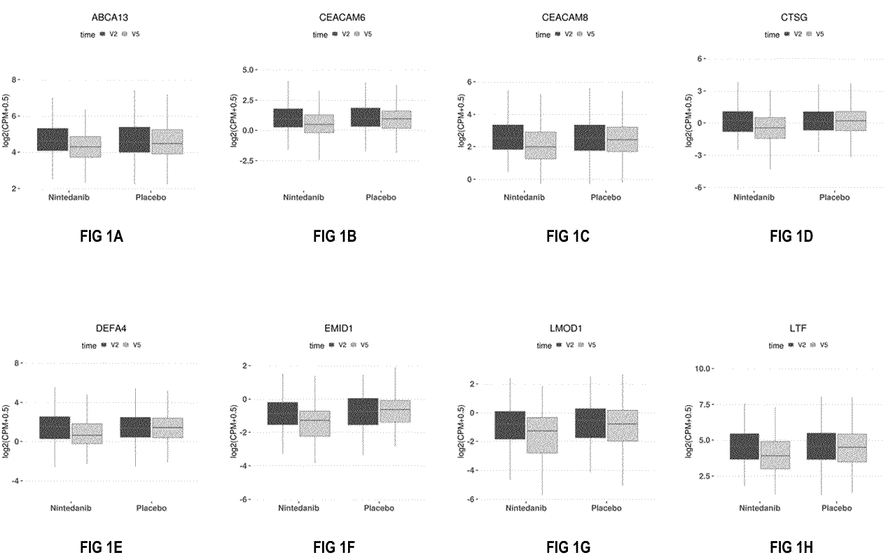

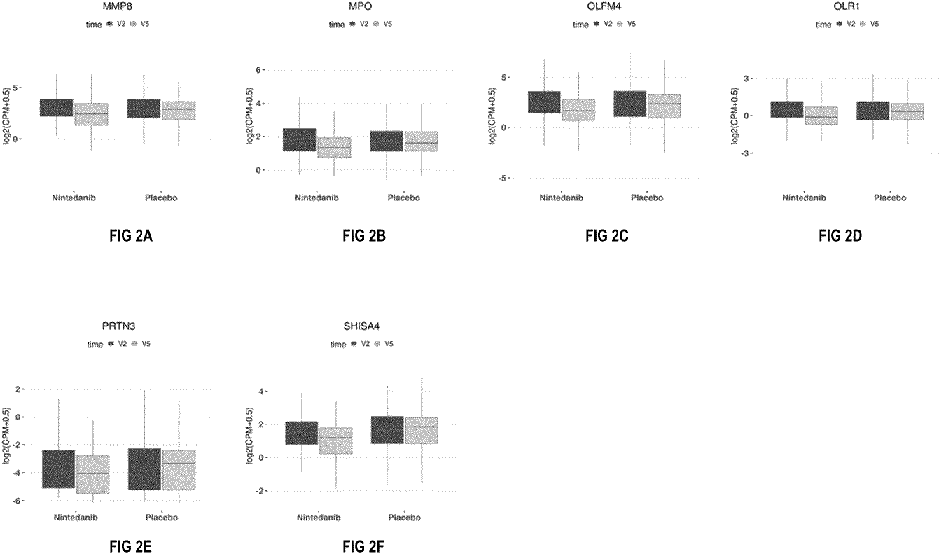

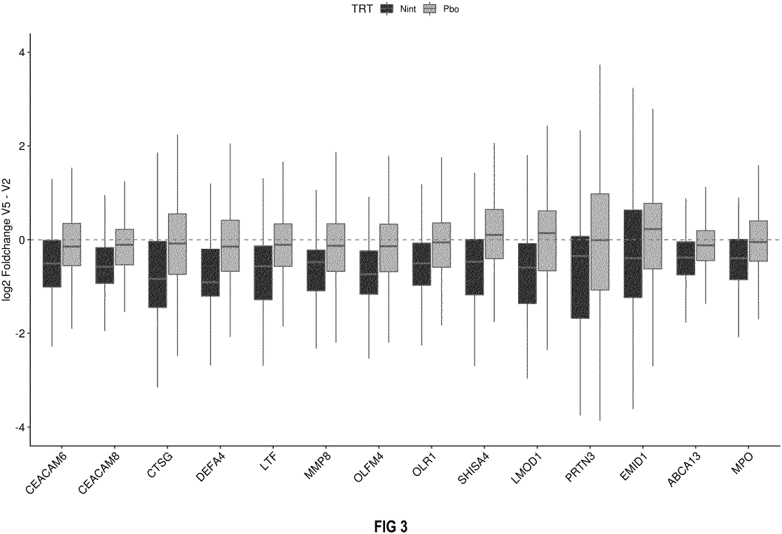

All of the 14 selected genes displayed a stronger reduction upon Nintedanib as compared to Placebo treatment, the latter of which only led to minor or no changes in gene expression (FIGS. 1 and 2). The finding that per-patient calculated fold changes (FIG. 3) showed similar and, in some instances, stronger effects, indicates that the observed grouped changes (FIGS. 1 and 2) are robust and not driven by outliers.

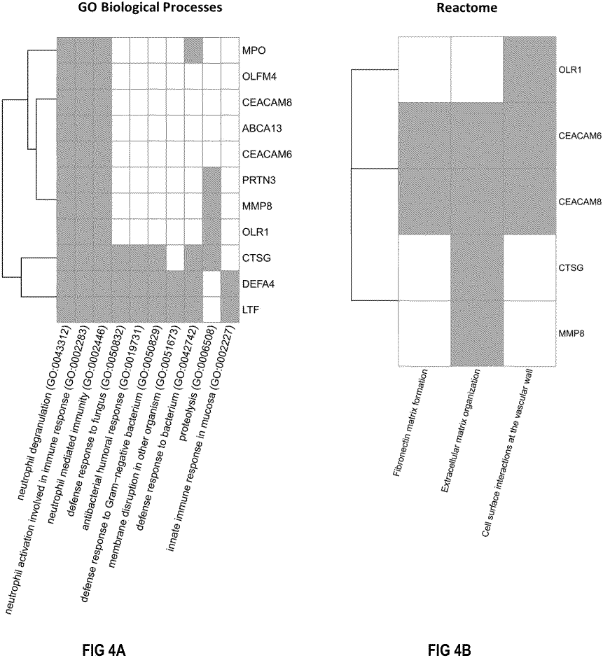

All of the 14 genes were found to show a highly significant association with ILD-relevant pathways, for example, 11 of the 14 genes (ABCA13, CEACAM6, CEACAM8, CTSG, DEFA4, LTF, MMP8, MPO, OLFM4, OLR1, PRTN3) are associated with the Gene Ontology (GO) Biological Process term “neutrophil mediated immunity (GO:0002446)” (adj. p-value 9.25E-13) and 4 of the 14 genes with the Reactome pathway “Extracellular matrix (ECM) organization Homo sapiens R-HSA-1474244” (adj. p-value 0.02687) (FIG. 4). CEACAM6 and CEACAM8 are also associated with the Reactome pathway “Fibronectin matrix formation Homo sapiens R-HSA-1566977” (adj. p-value 0.01043). LMOD1 was previously shown to be upregulated in IPF tissue vs. controls (Selman et al., Am J Respir Crit Care Med. 2006 Jan 15;173(2):188-98.) and EMID1 is a ECM/matrisome-associated protein. No known biological role was found for SHISA4. However, Shisa orthologues in mice were described as Wnt and Fgf signaling antagonists (Furushima et al., Dev Biol. 2007 Jun 15;306(2):480-92.). Moreover, four of the 14 genes (CEACAM6, CTSG, DEFA4, OLFM4) overlapped with an upregulated whole blood 13-gene set previously found to distinguish severe from mild IPF (Yang et al., PLoS One. 2012;7(6):e37708.) defined by differences in the diffusing capacity for carbon monoxide (DLCO) value. CTSG, OLFM4, DEFA4, CEACAM8 and LTF were further contained in a list of 20 genes that distinguished severe IPF from healthy controls (Yang et al., PLoS One. 2012;7(6):e37708.). DEFA4, OLFM4 and CTSG were further part of a whole blood 5-gene set that showed differential expression between primary myelofibrosis (PMF) and controls (Hasselbalch et al., PLoS One. 2014 Jan 13;9(1):e85567.). CEACAM6, CEACAM8, MPO and MMP8 were further part of a whole blood 7-gene set that differentiated prefibrotic myelofibrosis (prePMF) from essential thrombocytemia (ET) (Skov et al., PLoS One. 2016 Aug 31;11 (8):e0161570.).

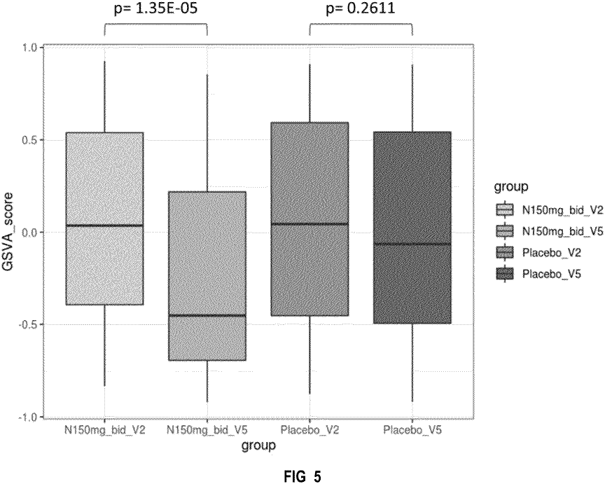

To assess the differential effect of Nintedanib versus Placebo treatment on the identified 14-gene set, non-parametric, unsupervised gene set variation analysis (GSVA) was performed (Hanzelmann et al., BMC Bioinformatics. 2013 Jan 16; 14:7.). The results show a highly significant reduction (p = 1.35E-05) in GSVA score upon Nintedanib treatment, whereas no significant changes were observed following treatment with Placebo (FIG. 5).

### Validation of Nintedanib-Induced Expression Changes in Non-IPF ILDs

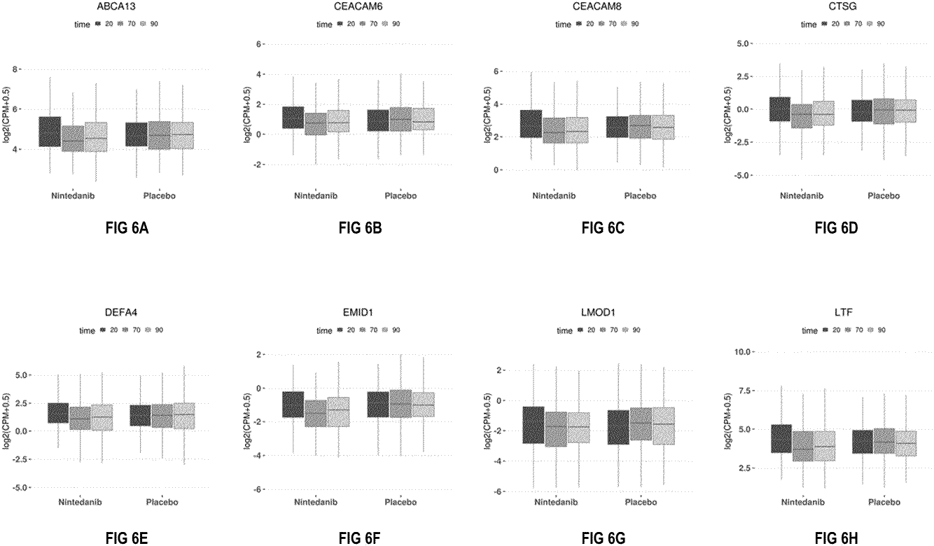

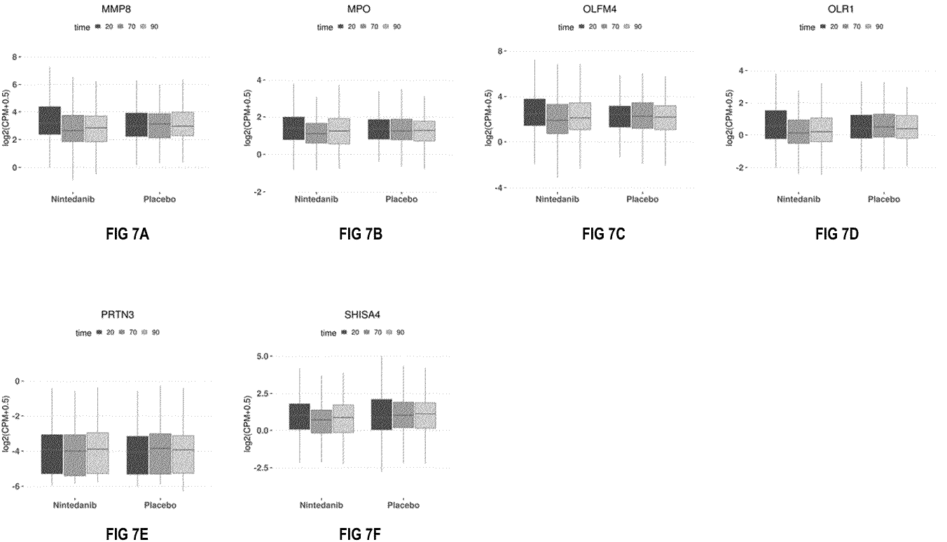

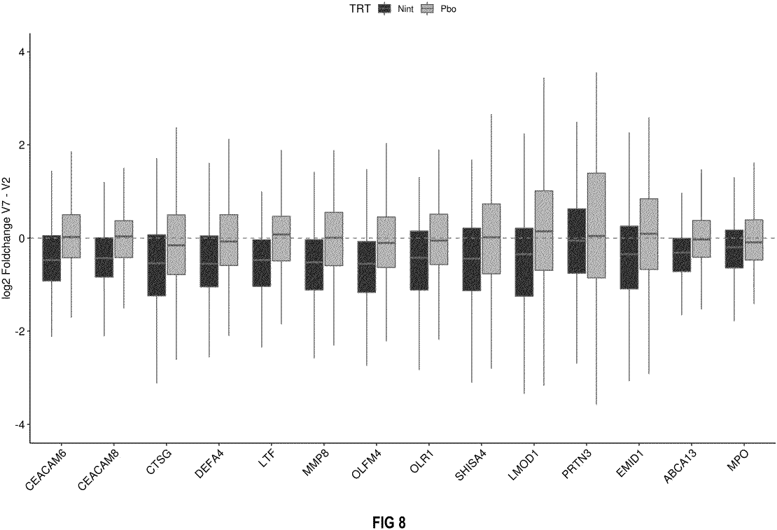

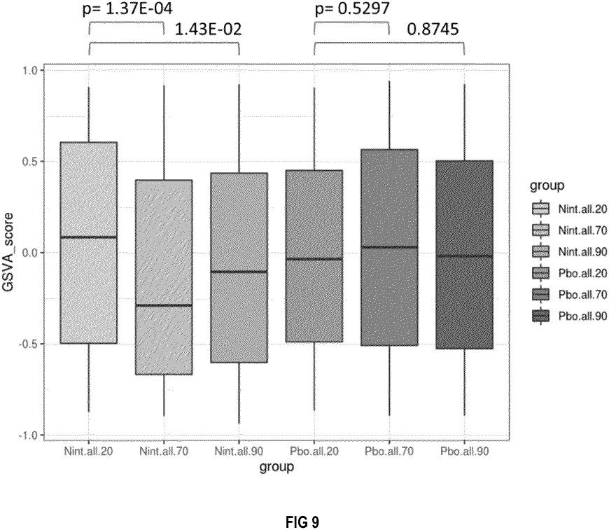

Following identification of the 14 Nintedanib-responsive genes in the INMARK trial in the context of IPF, expression of these genes was assessed in two additional clinical trials. The SENSCIS trial investigated the effect of Nintedanib versus Placebo treatment in a cohort of 580 patients with SSc-ILD over a time frame of 52 weeks. The PF-ILD trial INBUILD investigated the effect of Nintedanib versus Placebo treatment in a cohort of 662 patients with progressive fibrosing ILDs, including connective tissue disease (CTD)-associated ILD, rheumatoid arthritis-associated ILD (RA-ILD), chronic fibrosing hypersensitivity pneumonitis (HP), idiopathic non-specific interstitial pneumonia (iNSIP), unclassifiable idiopathic interstitial pneumonia (IIP), environmental/occupational lung disease or sarcoidosis over a time frame of 52 weeks. In both of these trials, whole blood samples were prospectively collected and the expression of the 14 said genes was retrospectively analyzed by whole-transcriptome RNA-sequencing. Consistent with the findings in the INMARK trial, most of the 14 genes were downregulated in the SENSCIS study upon Nintedanib treatment but not following Placebo, at 24 weeks (V70) and, in some instances, at 52 weeks (V90) post treatment (FIGS. 6 and 7). Similar effects were observed when the fold changes in expression were calculated in a per-patient fashion (FIG. 8). GSVA analysis further showed a significant reduction in GSVA score following Nintedanib treatment at both visits, whereas no significant changes were observed following treatment with Placebo (FIG. 9).

### C) Formulations for Oral Administration Containing Nintedanib

Soft gelatin capsules containing 150 mg of nintedanib

| | | Formulation A | Formulation B | Formulation C |
|---|---|---|---|---|
| Ingredients | Function | mg per capsule | mg per capsule | mg per capsule |
| Nintedanib monoethanesulphonate | Active Ingredient | 180.60 | 180.60 | 180.60 |
| Triglycerides, medium-chain | Carrier | 122.85 | 161.10 | 160.20 |
| Hard fat | Thickener | 114.75 | 76.50 | 76.50 |
| Lecithin | Wetting agent / Glidant | 1.80 | 1.80 | 2.70 |
| Gelatin | Film-former | 142.82 | 142.82 | 142.82 |
| Glycerol 85% | Plasticizer | 62.45 | 62.45 | 62.45 |
| Titanium dioxide | Colorant | 0.47 | 0.47 | 0.47 |
| Iron oxide A | Colorant | 0.08 | 0.08 | 0.08 |
| Iron oxide B | Colorant | 0.22 | 0.22 | 0.22 |
| Total Capsule Weight | | 626.04 | 626.04 | 626.04 |

Further pharmaceutical dosage forms for oral administration comprising nintedanib are disclosed in WO 2009/147212 and in WO 2009/147220.

1. A method for treating progressive fibrosing interstitial lung (PF-ILD) diseases in a patient in need of treatment, the method comprising
a) obtaining a first biological sample from the patient prior to the start of administering an antifibrotic agent;
b) administering or having administered an antifibrotic agent to the patient; wherein the antifibrotic agent is selected from:
(i) nintedanib, or its pharmaceutically acceptable salts, and
(ii) pirfenidone, wherein each antifibrotic agent is administered to the patient either as a monotherapy, or
in combination with one another, or
in combination with sildenafil or a pharmaceutically acceptable salt thereof,
c) obtaining a second biological sample from the patient after administering the antifibrotic agent;
d) measuring in said first and second sample the levels of expression of one or more biomarkers, selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof; and
e) comparing the levels of expression of said one or more biomarkers obtained from first and second biological samples, optionally taking into account control values.

2. A method for treating progressive fibrosing interstitial lung diseases in a patient in need of treatment, the method comprising
(a) administering an antifibrotic agent to the patient; wherein the antifibrotic agent is selected from:
(i) nintedanib, or its pharmaceutically acceptable salts, and
(ii) pirfenidone, wherein each antifibrotic agent is administered to the patient either as a monotherapy, or
in combination with one another, or
in combination with sildenafil or a pharmaceutically acceptable salt thereof,
b) obtaining a biological sample from the patient after administering the antifibrotic agent to the patient;
c) measuring in said sample the levels of expression of one or more biomarkers, selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof;
d) comparing the levels of expression of said one or more biomarkers compared with control values; and
e) determining whether or not the difference in the levels between the sample and the control reflects a beneficial response in the patient.

3. The method according to claim 1 wherein
the method further comprises the step of g1) continuing the administration of the antifibrotic agent to the patient or
g2) reducing the dose or dose frequency; if a beneficial response in the patient has been detected.

4. The method according to claim 1 2 wherein
the method further comprises the step of g3) discontinuing the administration of the antifibrotic agent to the patient or
g4) increasing the dose or dose frequency of the antifibrotic agent; if no beneficial response in the patient has been detected.

5. A method for treating progressive fibrosing interstitial lung diseases in patient in need of treatment, the method comprising
a) obtaining a biological sample obtained from the patient;
b) measuring in said sample the levels of expression of one or more biomarkers, selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof;
c) comparing the levels of expression of said one or more biomarkers with control values;
d) determining whether or not the difference between the expression levels in the sample and the control value reflects the need for initiation of a treatment of the fibrotic disorder; and
e) administering an antifibrotic agent to the patient if a need for initiation of a treatment of the fibrotic disorder has been determined, wherein the antifibrotic agent is selected from:
(i) nintedanib, or its pharmaceutically acceptable salts, and
(ii) pirfenidone, wherein each antifibrotic agent is administered to the patient either as a monotherapy, or in combination with one another, or in combination with sildenafil or a pharmaceutically acceptable salt thereof.

6. A method for selecting patients with progressive fibrosing interstitial lung diseases for treatment with antifibrotic agents, the method comprising
a) obtaining a biological sample from the patient prior to the start of administering the antifibrotic agent;
b) measuring in said sample the levels of expression of said one or more biomarkers;
c) comparing the levels of expression of said one or more biomarkers with control values;
d) determining whether the patient is eligible for treatment with antifibrotic agents on the basis of the results of the comparison, wherein the one or more biomarkers is selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof.

7. A method for using one or more biomarkers for predicting the progression of progressive fibrosing interstitial lung (PF-ILD) diseases in a patient comprising
a) obtaining a biological sample from the patient prior to the start of administering an antifibrotic agent to the patient;
b) measuring in said sample the levels of expression of said one or more biomarkers;
c) comparing the levels of expression of said one or more biomarkers with control values; and
d) predicting the progression of the disease on the basis of the results of the comparison, wherein the one or more biomarkers is selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof.

8. A method for using one or more biomarkers for determining whether an antifibrotic agent is efficacious in the treatment of progressive fibrosing interstitial lung (PF-ILD) diseases, in a patient in need of treatment comprising
a) obtaining a first biological sample from the patient prior to the start of administering the antifibrotic agent to the patient;
b) administering the antifibrotic agent to the patient;
c) obtaining a second biological sample from the patient after administering the antifibrotic agent;
d) measuring in said first and second sample the levels of expression of said one or more biomarkers; and
e) comparing the levels of expression of said one or more biomarkers obtained from first and second biological samples, optionally taking into account control values, wherein
the one or more biomarkers is selected from the group consisting of the genes CEACAM6, CEACAM8, CTSG, DEFA4, LTF, MMP8, OLFM4, OLR1, SHISA4, ABCA13, EMID1, LMOD1, MPO, PRTN3 and combinations thereof.

9. The method according to claim 1 wherein
the antifibrotic agent is selected from nintedanib monoethanesulphonate, optionally in combination with sildenafil citrate, and pirfenidone.

10. The method for according to claim 1 wherein
the PF-ILD is selected from the group consisting of idiopathic pulmonary fibrosis (IPF), systemic sclerosis-associated ILD (SSc-ILD), connective tissue disease-associated ILD (CTD-ILD), rheumatoid arthritis-associated ILD (RA-ILD), chronic fibrosing hypersensitivity pneumonitis (HP), idiopathic non-specific interstitial pneumonia (iNSIP), unclassifiable idiopathic interstitial pneumonia (IIP), environmental/occupational fibrosing lung disease, idiopathic pneumonia with autoimmune features (IPAF) and sarcoidosis.

11. The method according to claim 1 wherein at the start of administration of the antifibrotic agent, the patient shows a forced vital capacity of about 40% or more and/or a diffusing capacity of lung for carbon monoxide of about 40% or more.

12. The method according to claim 1 wherein the biological sample is a blood or peripheral blood mononuclear cell sample.

13. The method according to claim 1 wherein
the one or more biomarkers is selected from the group consisting of the genes CEACAM6, CEACAM8, CTSG, DEFA4, LTF, MMP8, OLFM4, OLR1 and combinations thereof.
